(19) United States
(12) Patent Application Publication
(10) Pub. No.: US 2004/0028622 A1
(43) Pub. Date: Feb. 12, 2004

(54) Multifunctional flavor systems and method of use

(76) Inventor: Michael Gurin, Glenview, IL (US)

Correspondence Address: Kajane McManus, P.O. Box 344, Wonder Lake, IL, 60097, US

(21) Appl. No.: 10/397,036

(22) Filed: Mar. 25, 2003

Related U.S. Application Data
(60) Provisional application No. 60/402,569, filed on Aug. 12, 2002.

(51) Int. Cl. A61K 9/68; A61K 7/16

(52) U.S. Cl. 424/48; 424/49

(57) ABSTRACT

A composition and method for functionalizing confectionery, chewing gum, oral care and beverage products by intensifying the flavor using ingredients comprised of flavor potentiators, enhancers, and amplifiers. The composition is further comprised of oral care actives selected to control halitosis and dental plaque utilizing polyphenols and enzymes whose activity levels are protected by stabilization methods. Such stabilization methods enhance and prolong the flavor release characteristics of such products.

### CROSS-REFERENCE TO RELATED APPLICATIONS

This application claims priority from U.S. Provisional Patent Application Serial No. 60/402,569 filed Aug. 12, 2002 and entitled CONFECTIONERY COMPOSITIONS AND METHOD OF USE.

### BACKGROUND OF THE INVENTION

The present invention relates to the field of chewing gum, oral care, confectionery, and beverage compositions and in particular to compositions that incorporate flavor additives, and oral care actives for consumer use and to enhance and prolong the flavor release characteristics of such products. The flavor additives are comprised of additives that intensify flavor due to physical, and enzymatic modifications. The oral actives are comprised of protected polyphenols and enzymes to control halitosis and dental plaque. The inventive additives and actives synergistic roles as flavor enhancers address many of the weaknesses of present day chewing gum, confectionery, oral care, and beverage compositions.

One such weakness is highlighted within the specific application of chewing gum. Chewing gum is comprised principally of gum base, flavor additives, emulsifiers, and sweeteners. Gum bases known in the art are virtually incompatible with high levels of fats and oils, thus preventing the development of a wide range of chewing gum flavors. As a result, the creation of flavors typically associated with fats or creams (e.g., chocolate, peanut butter, almond butter, vanilla, etc.) are manufactured using only extracts, or artificial flavors. The result is a complete absence of mouthfeel and real taste profiles.

The drive for low fat foods, specifically in beverages and confectionery products, faces many of the same challenges as chewing gum, but for health reasons as compared to fat compatibility reasons. The drive to incorporate many additional functional actives, proteins, and fiber into confectionery and beverage products also accentuates the drive to “free up” space by further reducing the material loading of non-functional ingredients, such as fats, sugars, sugar alcohols, and artificial flavors. Reduction of these components places further burden on other ingredients, actives, and additives to achieve desirable fat mouthfeel, flavor masking of many functional ingredients, and flavor amplification of desirable flavors.

U.S. Pat. No. 5,223,283 discloses the use of between about 0.3 and about 2.4 percent cocoa powder with the specific intent of not contributing a chocolate flavor to the gum base and solely for the purpose of making synthetic gum base taste more like a natural gum base.

U.S. Pat. No. 4,889,726 discloses the use of cocoa powder between about 0.08 and 0.5 percent by weight of the chewing gum as a method of enhancing the flavor of a mint flavored chewing gum, again with the specific intent of not contributing a chocolate flavor to the gum base.

Protein hydrolysate and its derivatives; enzymatically modified protein powder and hydrolysate products; and fermented protein products are currently used as savory components in meat, poultry, baked goods, dressings, seafood, soups, dry mixes and seasoning blends.

The present invention provides a new, optimal and low cost composition and method of use, which achieves superior performance and new applications over the above-referenced prior art, and others.

### SUMMARY OF THE INVENTION

As used herein, the term “confectionery products” includes cookies, bars, fudge, caramels, taffy, mints, chocolates, butters, spreads, chewing gum, and gummy products.

As used herein, the term “beverage products” includes dry powder mixes, carbonated drinks, dairy milk and yogurt drinks, non-dairy milk and yogurt drinks, chocolate drinks, coffees, and teas.

As used herein, the term “oral care products” includes chewing gum, mints, toothpaste, mouthwash, breathstrips, edible films, liquid concentrate drops, and soft capsules.

As used herein, the term “new flavor profiles” refers to a chewing gum composition that incorporates flavors from oils, fats, creams and fatty acids of mono-, di-, and tri-glycerides derived from vegetable oils, nut oils, dairy products, and bean products.

As used herein, the term “flavor profiles of increased intensity” refers to flavor notes that are amplified in comparison to non-modified flavor raw materials, herein referred to as amplified flavors. Amplified flavors accentuate the same flavor note as itself beyond the level of non-amplified flavor additives.

As used herein, the term “flavor potentiators” refers to flavor additives that potentiate different flavor notes than themselves beyond the level of non-potentiated flavor additives.

As used herein, the term “flavor amplified composition” or hereinafter referred to as “FACs” refers to the collective group of chewing gum, confectionery, oral care, and beverage compositions incorporating the inventive flavor amplifiers, flavor potentiators, and synergistic oral care actives.

As used herein, the term “lipolyzed or enzymatically modified triglycerides”, hereinafter referred to as “LEMT”, includes enzymatically modified oils, fats, and fatty acids of mono-, di-, and tri-glycerides, and lipolyzed modified oils, fats, and fatty acids of mono-, di-, and tri-glycerides.

As used herein, the term “water activity” is defined as the measurement of how tightly water is bound physically vs. chemically inside a food product.

The inventive chewing gum or oral care compositions achieve increased intensity flavor profiles by amplifying flavor additives with enzymatically modified oils, fats, and fatty acids of mono-, di-, and tri-glycerides; lipolyzed modified oils, fats, and fatty acids of mono-, di-, and tri-glycerides; and pervaporated flavors, and employ standard conventional methods for compounding chewing gum or oral care compositions as are known in the art.

The inventive chewing gum or oral care compositions achieve increased intensity flavor profiles by including protein hydrolysate and its derivatives; enzymatically modified protein powder and hydrolysate products; and fermented protein products with standard conventional methods for compounding chewing gum or oral care compositions as are known in the art with flavor potentiators.

The inventive confectionery or beverage compositions achieve increased intensity flavor profiles by combining both flavor potentiators and amplified flavor additives while employing standard conventional methods for compounding confectionery or beverage compositions as are known in the art. The flavor potentiators include protein hydrolysate and its derivatives; enzymatically modified protein powder and hydrolysate products; and fermented protein products. The amplified flavor additives include enzymatically modified oils, fats, and fatty acids of mono-, di-, and tri-glycerides; lipolyzed modified oils, fats, and fatty acids of mono-, di-, and tri-glycerides; and pervaporated flavors.

In accordance with another aspect of the present invention, the amplified flavor additives are present in an amount up to approximately 10% on a total weight basis.

In accordance with another aspect of the present invention, the amplified flavor additives are lipolyzed or enzymatically modified additives, preferably selected from raw materials in the group of cocoa butter, peanut oils, dairy fats and creams, vegetable oils, nut oils, and other triglycerides.

In accordance with yet another aspect of the present invention, the dairy fats and creams, and other triglycerides are more preferably selected from raw materials in the group of butter, cheese, milk, natural oils from spices, and fruits.

In accordance with another aspect of the present invention, the flavor potentiators are preferably derived from raw materials selected from the group of soy, wheat, and dairy proteins.

In accordance with another aspect of the present invention, the confectionery, oral care, or beverage products are further comprised of fat mimetics.

In accordance with yet another aspect of the present invention, the fat mimetics are preferably selected from the group of cocoa, partially defatted nut flour, defatted nut flour, food starch, modified food starch, protein, liposome, chitosan, peptide, fiber, cellulose, lecithin and interesterified triglycerides. The more preferable fat mimetics as referenced herein are physically modified to modify the release of flavors and to achieve an enhanced fat mouthfeel. The more specifically preferred fat mimetics both emulate and provide fat mouthfeel and flavor.

In accordance with the invention, the fat mimetic is preferably reduced in size to particles having an average size of less than approximately 10 microns.

In accordance with the invention, the fat mimetic is more preferably reduced in size to particles having an average size from approximately 100 nanometers to approximately 5000 nanometers.

In accordance with the invention, the fat mimetic is specifically reduced in size by at least one process selected from the group of sonication, hydrodynamic cavitation, and cryogenic grinding.

In accordance with another aspect of the invention, the confectionery, oral care, or beverage products are further comprised of oils and fats in triglyceride form processed by enzymatic interesterification to obtain products free of trans-fatty acids and with special physical and chemical characteristics. The enzymatic interesterification process can create a random distribution, or it may be directed to a degree that actually modifies the shortening properties, without increasing saturation or creating trans isomers.

In accordance with yet another aspect of the present invention, the oils and fats being enzymatic interesterified are preferably selected oils and fats in triglyceride form having a minimum of approximately 10% on a weight basis of one or more of omega-3 and omega-9 polyunsaturated fatty acids, eicosapentaenoic acid, “EPA”, and docosahexaenoic acid, “DHA”.

In accordance with another aspect of the present invention, the confectionery, oral care, or beverage products are further comprised of a fruit concentrate sweetener as humectant that comprises a blend of hydrolyzed starch having a dextrose equivalent (D.E.) of up to 25; fruit juice or fruit syrup concentrate of at least approximately 40% soluble solids and 0% insoluble solids thereby forming a liquor having a dry weight composition of approximately 40 to 65% complex carbohydrates; approximately 35 to 55% simple sugars from the fruit juice or fruit syrup concentrate; and approximately 0 to 5% nutritional components occurring naturally in the fruit juice or fruit syrup concentrate. The fruit concentrate sweetener composition further solubilizes the glycerides selected from at least one of enzymatically modified oils, fats, and fatty acids of mono-, di-, and tri-glycerides, and lipolyzed modified oils, fats, and fatty acids of mono-, di-, and tri-glycerides.

In accordance with yet another aspect of the present invention, the fruit concentrate sweetener composition is further processed with steps selected from the group of drying to approximately 78 to 80% soluble solids to make the sweetener composition suitable for replacing corn; and drying to approximately 96 to 99% soluble solids to make the sweetener composition suitable for replacing powdered or granulated sucrose.

Yet another aspect of the present invention is the further inclusion, within the confectionery, oral care, or beverage product, of at least one oral care active selected from the group of encapsulated, colloidal stabilized, or complexed polyphenols; enzymes stabilized against reduction in activity and protein denaturing, such enzymes selected from the group of dental plaque inhibiting, halitosis inhibiting, and digestive aids; natural antioxidants, such antioxidants specifically limiting formation of free radicals in the oral cavity; and metal chelators and bicarbonate.

Another aspect of the present invention is the further inclusion, within the confectionery, oral care, or beverage product, of at least one additional flavor ingredient selected from partially denatured protein; and monoammonium glycyrrhizinate. Partially denatured protein enables complexation with polyphenols in order to reduce the astringent taste. Monoammonium glycyrrhizinate more preferably reduces the astringent taste especially with cocoa powder and cocoa polyphenols.

Yet another aspect of the present invention is the further inclusion of a combination of at least one water-soluble and at least one oil soluble polyphenol in order to reduce both hydrophilic and hydrophobic bacteria from the oral cavity.

In accordance with the present invention, the polyphenols are preferably selected from polyphenol extracts of cocoa, cocoa bean husk, apple, grape, and tea.

Another aspect of the present invention is the further mixing of polyphenols with citric acid in order to prevent the ionization of the hydroxy groups of polyphenols.

Yet another aspect of the present invention is the further inclusion of bioadhesive agents to further promote the binding to mucin and mucosa of at least one or a blend of polyphenols and enzymes.

Another aspect of the present invention is the further compounding of polyphenols and enzymes with at least one encapsulant selected from the group of sugar esters, liposomes, casein and calcium phosphate, matrix of starch hydrolysate acid ester, and matrix of hydrogenated starch hydrolysate and maltodextrin.

Yet another aspect of the present invention is the colloidal stabilization of polyphenols to reduce polyphenol complexation and precipitation by the further inclusion of at least one stabilizer selected from the group of polysaccharides, and citric acid.

Another aspect of the present invention is the complexation of polyphenols by at least one complexing agent selected from the group of dairy proteins, and partially denatured proteins.

In accordance of the present invention, the active enzymes are stabilized in order to reduce the loss of activity and protein denaturing by further inclusion of at least one stabilizer selected from the group of lactoperoxidase, pure grain alcohol, polysaccharides, and block-wise enzymatically de-esterified pectin, sugar esters, liposomes, starch hydrolysate, and protein hydrolysate.

Yet another aspect of the present invention is to preferably select polysaccharides from the group of gum arabic, and pectin in order to stabilize and protect polyphenols and enzymes from loss in activity levels.

Encapsulation is powerful in enhancing the performance of the amplified flavors, flavor potentiators, and oral care actives in accordance with the present invention. The encapsulant shell according to the present invention is preferably comprised of at least one from the group of gum arabic, fat mimetics, liposomes, liposomes in sol-gels, shellac, fats, hydrolyzed fats, ethyl cellulose, hydroxy propyl methylcellulose, starches and modified starches, polymers, waxes, alginate and alginic acid (e.g., sodium alginate), calcium caseinate, calcium polypectate, carboxyl cellulose, carrageenan, cellulose acetate phthalate, cellulose acetate trimellitate, chitosan, corn syrup solids, dextrins, fatty acids, fatty alcohols, gelatin, gellan gums, hydroxy cellulose, hydroxy ethyl cellulose, hydroxy methyl cellulose, hydroxy propyl cellulose, hydroxy propyl ethyl cellulose, hydroxy propyl methyl cellulose, hydroxy propyl methyl cellulose phthalate, lipids, liposomes, low density polyethylene, mono-, di- and tri-glycerides, pectins, phospholipids, polyethylene glycol, polylactic polymers, polylactic co-glycolic polymers, polyvinyl pyrolindone, stearic acid and derivatives, gums (e.g., xanthum) and proteins (e.g., zein, gluten).

Numerous means of creating encapsulations are known in the industry, whereby the flavor additives, flavor potentiators, oral care actives, and amplified flavor additives are encapsulated, and incorporated in the present invention including subjecting encapsulated ingredients to methods selected from the group of dropping method filler, spray dried, fluid-bed coated, coacervation, molten encapsulating with spray chilling, and mixing flavor enhancer with a polymer as the encapsulating agent with the resulting mixture being extruded into fine fibers, mixed with an absorbent, and included in a powder material.

In accordance with the present invention, encapsulated ingredients are further comprised of at least one from the group of glycerides selected from enzymatically modified oils, fats, and fatty acids of mono-, di-, and tri-glycerides; glycerides selected from lipolyzed modified oils, fats, and fatty acids of mono-, di-, and tri-glycerides; pervaporated flavors; high intensity sweeteners; ingredients selected from the group of colorants, nutraceutical actives, pharmaceutical actives and antioxidants; salt or salt substitutes; prior encapsulated actives; and standard flavors. The utilization of multiply encapsulated flavor additives, flavor potentiators, oral care actives, and amplified flavor additives is in accordance with and preferred in the present invention. The multiply encapsulated shell is further comprised of alternating shells made from water or oil soluble ingredients that further encapsulate at a minimum the prior encapsulated actives.

The more specifically preferred encapsulation is further comprised of active release agents to achieve controlled release of flavor, pharmaceutical, or nutraceutical actives; bioadhesive agents to bind encapsulant shell and encapsulated actives to mucin and mucosa; and time release agents to regulate the dissolution, melting, or diffusion of the shell. The encapsulated shell is designed to achieve release of the encapsulated flavor ingredients by at least one mechanism selected from the group of mechanical rupture of the capsule shell, dissolution of the shell, melting of the shell, and diffusion of the shell.

In accordance with the present invention, optimizing the performance of all flavor and oral care actives is achieved by encapsulation in addition to specific means of protecting the oral care actives. Specific means of protecting the oral care actives are preferably selected from incorporating such enzymes through at least one from the group of blending with a powdered gum base; maintaining process temperatures below enzyme deactivation or protein denaturing temperatures; applying active enzymes as a coating after any high temperature processing, such high temperature processing being accomplished above enzyme deactivation or protein denaturing temperatures; applying active enzymes as blended ingredient after any high temperature processing, such high temperature processing being accomplished above enzyme deactivation or protein denaturing temperatures; applying active enzymes as a liquid center of confectionery to minimize exposure to high temperature processing, such high temperature processing being accomplished above enzyme deactivation or protein denaturing temperatures; utilizing encapsulation of active enzymes able to withstand high pressures of stamping or tabletting; utilizing multilayer encapsulation of active enzymes; utilizing enzymes in a liquid form to achieve maximum activity levels; and minimizing encapsulant shell diameter size to less than approximately 2 millimeters of encapsulated active enzymes.

Advantages of the present invention include providing a chewing gum composition that introduces new flavor profiles and increases the intensity of existing flavor profiles while mimicking the texture and chew properties of standard chewing gum.

Another advantage of the present invention is a controlled flavor release over an extended duration.

Yet another advantage of the present invention is that a lower level of flavor may be used compared to usage levels in conventional confectionery, oral care, and beverage products.

A still further advantage of the present invention is that enzymatically modified oils, fats, and fatty acids of mono-, di-, and tri-glycerides, and lipolyzed modified oils, fats, and fatty acids of mono-, di-, and tri-glycerides are further solubilized as compared to conventional confectionery, oral care, and beverage products.

Yet another advantage of the present invention is the standard texture and mouthfeel associated with non-triglyceride flavorants throughout the taste experience.

Another advantage of the present invention is to provide flavored and actives granules that can be simply mixed into powdered chewing gum base to ensure enzyme and protein effectiveness.

A still further advantage of the present invention is to provide a method of encapsulating the flavor and actives granules to achieve time release of the flavor.

Additional features and advantages of the present invention are described in and will be apparent from the detailed description of the presently preferred embodiments. It should be understood that various modifications to the presently preferred embodiments described herein will be apparent to those skilled in the art. Such modifications can be made without departing from the spirit and scope of the present invention and without diminishing its attendant advantages. It is therefore intended that such modifications be covered by the appended claims.

### DETAILED DESCRIPTION OF THE PREFERRED EMBODIMENTS

The present invention provides improved chewing gum, confectionery, oral care, and beverage compositions including flavor enhancers and methods for making same. Flavor enhancers are used to amplify and accentuate traditional flavors incorporated into chewing gum, confectionery, oral care, and beverage compositions. The utilization of flavor additives modified by a range of physical, enzymatic, fermentation, and chemical processes is one such method of amplifying flavor intensity. Another method of amplifying flavor intensity is through the utilization of fat mimetics, with functionality differing from the traditional emulation of fat mouthfeel, as a synergistic flavor delivery component. The integration of the inventive flavor additives into the chewing gum, confectionery, oral care, and beverage compositions is further enhanced through the use of a multipurpose additive that provides a unique combination of benefits that include humectant, flavor enhancer, and fat solubilizer. Many of the flavor additives that specifically amplify the flavor intensity are of a family of enzymes or polyphenols that further exhibit secondary benefits in the area of oral care. The unique ability to integrate the enzymes or polyphenols as both flavor amplifiers and functionally active oral care additives negates the often associated disadvantage of chewing gum, confectionery, oral care, and beverage compositions of causing dental plaque. Lastly, delivery of the inventive additives and actives is best achieved through optimization methods, such as incorporating protection of additives and actives against reduced activity levels.

Flavor Amplifiers

Chewing gum or oral care compositions achieve increased intensity flavor profiles by amplifying flavors through the inventive additives of enzymatically modified oils, fats, and fatty acids of mono-, di-, and tri-glycerides; lipolyzed modified oils, fats, and fatty acids of mono-, di-, and tri-glycerides; and pervaporated flavors, while employing standard methods for compounding chewing gum or oral care compositions as are known in the art.

The addition of said amplified flavor additives is in an amount up to approximately 10% on a total weight basis. More preferred usage rates are between approximately 0.2% to approximately 5% on a total weight basis. More specifically preferred usage rates are between approximately 0.2% and approximately 3% on a total weight basis. The usage rates are based on obtaining the desired flavor profile with secondary consideration to fat levels for nutritional and dietary purposes. Fat levels are determined in a subset of confectionery compositions, most specifically chewing gum, as a function of compatibility and mouthfeel performance. Chewing gum requires usage levels of fats less than approximately 10% due to otherwise excessive emulsification of the gum base.

The present invention provides FACs and methods for making same. Pursuant to the present invention, FACs are provided that includes enzymatically modified triglycerides. Fatty acids of mono-, di-, and tri-glycerides are liberated by enzymatic modification and reduction processes or by being lipolyzed. The resulting products are hereinafter referred to as lipolyzed or enzymatically modified triglycerides (“LEMT”). LEMTs are then subsequently microencapsulated and optionally combined with other ingredients prior to being manufactured into a chewing gum, confectionery, oral care, or beverage product. The importance of LEMTs to FACs are numerous with the primary objective of imparting desirable mouthfeel, enhanced flavor, and providing intensified fatty flavor notes while avoiding the excessive emulsifying effect traditionally associated with triglycerides. The presently preferred embodiments of the invention will now be set forth.

Due to the use of the LEMT, a FAC is provided that can provide the standard texture and mouthfeel associated with non-triglyceride flavorants throughout the chewing experience. Still further, the LEMT can be encapsulated to enable the time release and delivery of these flavorants to increase the period of consumer satisfaction. To this end flavors, colorants, drugs or nutritional compounds can be attached/encapsulated and then mixed into the base portion to become a FAC for the consumer.

As noted, the LEMT may be added to typical flavor formulas as supplemental flavors or used itself as the flavorant. Additionally, the ingredients of the flavor formulations may be further modified to provide desirable characteristics.

A composition and method for supplementing a water-soluble portion and a water insoluble portion including enzymatically modified fats, oils, and generally triglycerides in chewing gum systems is also contemplated. The composition is selected from the group of (A) enzymatically modified oils, fats, and fatty acids of mono-, di-, and tri-glycerides, (B) lipolyzed modified oils, fats, and fatty acids of mono-, di-, and tri-glycerides, (C) an encapsulating agent, and (D) standard chewing gum, confectionery, oral care, or beverage ingredients. The composition provides an effective increase in new flavor profiles, superior mouthfeel, acceptance, nutritional, therapeutic, and pharmacological properties in food, nutrition and diet systems through consumption.

Cocoa butter is processed with lipase enzymes to free the flavorful fatty acids from their triglyceride bond, resulting in a cocoa butter that provides a stronger flavor sensation. Lecithin is added as an emulsifier. Enzymes are then deactivated and encapsulated to make a dry powder.

The present invention does not preclude the use of enzymatically modified cocoa butter, as above, combined with additional ingredients prior to encapsulation. Preferred additional ingredients are selected from the group of dry milk powder and chocolate solids when the desired flavor is chocolate.

Butter is processed with lipase enzymes to free the flavorful fatty acids from their triglyceride bond, resulting in a butter that provides a stronger flavor sensation. Enzymes are then deactivated and encapsulated to make a dry powder.

Cheese is processed with lipase enzymes to free the flavorful fatty acids from their triglyceride bond, resulting in a cheese that provides a stronger flavor sensation. Enzymes are then deactivated and encapsulated to make a dry powder.

The present invention does not further preclude the use of enzymatically modified cheese being combined with additional ingredients prior to encapsulation. A preferred additional ingredient is spray dried beer.

Numerous snack foods utilize flavors that are selected from the group of hot spices, popcorn, vanilla, cinnamon, fruit, and other known flavors. Natural oils derived from many foods are also processed with lipase enzymes to free the flavorful fatty acids from their triglyceride bond, resulting in a flavor essence that provides a stronger flavor sensation.

Nut oils and meats are processed with lipase enzymes to free the flavorful fatty acids from their triglyceride bond, resulting in a nut flavor that provides a stronger flavor sensation. Preferred nut oils and meats are selected from the group of peanut, almond, pistachio, hazelnut, cashew, walnut, hemp seed and macadamia. Enzymes are then deactivated and encapsulated to make a dry powder.

The present invention does not preclude the use of enzymatically modified cocoa butter, butter, cream, cheese and all other LEMT flavors combined with additional ingredients prior to encapsulation. Preferred additional ingredients are selected from the group of dry milk powder, food ingredients, sweeteners, emulsifiers, softeners, colorants, nutraceutical actives, pharmaceutical actives and antioxidants.

The water soluble coating of the spray dried material envelops the LEMT and thus facilitates distribution thereof in the aqueous phase so that this method of incorporating the flavoring component of the invention results in uniform, stable emulsions which are not readily produced when the LEMTs are used in a liquid form. The water-soluble coating or carrier may be a conventional carrier such as non-fat dry milk, starches, corn syrup solids or maltodextrins. Starch hydrolysates having a lower dextrose equivalent “D.E.” are particularly suitable since they are readily water soluble and not too hygroscopic. When the D.E. is approximately 20 or less, such carriers are referred to as maltodextrins.

The modified triglycerides or oils include natural dairy, chocolate, nut, corn, and oil flavors, that can be obtained by incubating either milk-fat, cocoa butter, nut and oil triglycerides, and other vegetable triglycerides derived directly from whole milk, butter oil, vegetable oils, and nut oils derived from the oils with a lipase (esterase) enzyme.

The chewing gum is flavored by incorporating therein a sufficient quantity of oil soluble lipolyzed triglycerides (obtained by treating triglycerides with lipase enzymes) and water-soluble starter distillate. The proportion of water-soluble component to oil soluble component should be approximately 1 part water-soluble component per each 2-20 parts oil soluble component. It is necessary for the water phase to be distributed throughout the oil phase. This can be accomplished either by mixing the oil and water phases with a carrier such as maltodextrin and drying, which causes the phases to become encapsulated, or by emulsifying the two phases by means of an emulsifying agent. The final LEMT product exhibits the following characteristics:

It has the flavor intensity of approximately 10 grams of original triglyceride.

It has the natural flavor of triglyceride.

It dissolves instantly upon contact with wet and hot foods, thus providing not only the flavor but also the mouthfeel of triglyceride.

It has an extended shelf-life and is extremely stable to oxidation.

The utilization of amplified flavor additives is one method of emulating the performance of fats, specifically the ability to provide richness, creaminess, and mouthfeel. The utilization of lipolyzed or enzymatically modified additives preferably selected from raw materials in the group of cocoa butter, peanut oils, dairy fats and creams, vegetable oils, nut oils, and other triglycerides achieve the desired taste and mouthfeel benefits while minimizing the emulsification and total fat content. More preferred lipolized or enzymatically modified additives are selected from raw materials in the group of cocoa butter, nut oils, dairy fats and creams, and other triglycerides. Even more specifically preferred lipolyzed or enzymatically modified additives are from raw materials in the group of cocoa butter, peanut oil, butter, cheese, milk, natural oils from spices, and fruits.

The present invention does not preclude the direct addition of LEMT to the base product. The compositions of the present invention are thus processed at a temperature ideally below 80 degrees Celsius to prevent premature degradation or volatilization of the flavorant. Typical base product ingredients can then be blended and mixed with the flavor ingredients with little difficulty.

Flavor Potentiators

Chewing gum or oral care compositions achieve increased intensity flavor profiles by including the inventive flavor potentiators: protein hydrolysate and its derivatives; enzymatically modified protein powder and hydrolysate products; and fermented protein products and employing standard conventional methods for compounding chewing gum or oral care compositions as are known in the art. Protein hydrolysate and its derivatives; enzymatically modified protein powder and hydrolysate products; and fermented protein products more specifically potentiate cocoa, nut meats and oils, and mint oils. The flavor potentiators are preferably derived from raw materials selected from the group of soy, wheat, and dairy proteins. Furthermore, the mouthfeel further enhances the richness and creaminess of chewing gum and oral care compositions.

Confectionery or beverage compositions achieve the increased intensity flavor profiles desired by incorporating the inventive combination of both flavor potentiators and amplified flavor additives, and by standard manufacturing methods of compounding into confectionery or beverage compositions. The flavor potentiators include protein hydrolysate and its derivatives; enzymatically modified protein powder and hydrolysate products; and fermented protein products. The amplified flavor additives include enzymatically modified oils, fats, and fatty acids of mono-, di-, and tri-glycerides; lipolyzed modified oils, fats, and fatty acids of mono-, di-, and triglycerides; and pervaporated flavors. Furthermore, the mouthfeel further enhances the richness and creaminess of the confectionery and beverage compositions beyond their individual respective use.

Fat Mimetics

A plethora of fat mimetics exist as fat replacements in conventional foods, especially in traditionally high fat products such as margarine, baked products, and meat products. The inventive practice combines the use of fat mimetics with flavor potentiators and amplifiers to yield a rich, full, and rounded taste profile as compared to fat mimetics alone that have no positive effects on flavor or flavor release. Confectionery, oral care, or beverage products are further comprised of fat mimetics. The fat mimetics are preferably selected from the group of cocoa, partially defatted nut flour, defatted nut flour, food starch, modified food starch, protein, liposome, chitosan, peptide, fiber, cellulose, lecithin and interesterified triglycerides. More preferred interesterified triglycerides have the fatty acids in the 1- and 3-positions rearranged by 1,3-specific lipase enzymes. This results in more natural fats (i.e., without trans fatty acids) and fats that have superior mouthfeel than the non-interesterified counterparts. Specifically preferred interesterified triglycerides are selected from group of oils rich omega-3, and omega-9 polyunsaturated fatty acids, and eicosapentaenoic acid and docosahexaenoic acid. Interesterified triglycerides are more preferably selected where the oil has a minimum of approximately 10% on a weight basis of one or more of omega-3 and omega-9 polyunsaturated fatty acids, eicosapentaenoic acid and docosahexaenoic acid for confectionery and beverage compositions that are considered heart healthy products. This results in a wide range of nutraceutical and functional benefits while enhancing the flavor release and profile characteristics.

Many of the aforementioned fat mimetics themselves have no inherent fat mimetic qualities, specifically cocoa, partially defatted nut flour, defatted nut flour, protein, chitosan, peptide, fiber, and cellulose. However, the physical modification of such fat mimetics modifies the release of flavors and achieves an enhanced fat mouthfeel. A physically modified cocoa powder or defatted peanut flour provides a fat mouthfeel and flavor identical to the selected flavor. Utilizing physically modified fat mimetics such as cocoa powder, though not limited thereto, further reduces the required levels of fat mimetics and flavor amplifiers that are incorporated for mouthfeel. The reduction of fat levels is especially important in chewing gum compositions due to the emulsifying effect. A reduction of fat levels in beverages and confectionery compositions reduces the required levels of omega-3 and omega-9 polyunsaturated fatty acids, eicosapentaenoic acid and docosahexaenoic acid, all ingredients that are especially susceptible to rancidity.

The preferred physical modification of the aforementioned fat mimetics that have no inherent fat mimetic qualities is by particle size reduction. The more preferred particle size is an average size of less than approximately 10 microns. More specifically preferred particle size is an average size from approximately 100 nanometers to approximately 5000 nanometers. Particularly preferred fat mimetics with no inherent fat mimetic qualities are processed by at least one process or method selected from the group of sonication, hydrodynamic cavitation, and cryogenic grinding. All of these processes maintain low processing temperatures thereby maintaining all of the flavor volatiles present, and limiting the denaturing of proteins. A wide range of processing media is available for the particle size reduction. Powders that are ultimately utilized in a dry form are preferably processed in liquid gases such as nitrogen or carbon dioxide. Less preferable is the processing in liquid such as water or food grade solvents (e.g., ethanol) whereby cavitation is the size reduction method achieved by sonication or hydrodynamic cavitation. Subsequent drying follows the size reduction process that includes, though is not limited to, spray drying, vacuum drying, and freeze drying. Low temperature drying is preferable where susceptible flavor volatiles or proteins are present.

Solubilizer Enhancer

A secondary object of the present invention is to provide a FAC with natural flavors obtained from the inclusion of a fruit concentrate sweetener composition. The fruit concentrate sweetener composition comprises a blend of a hydrolyzed starch having a dextrose equivalent (D.E.) of up to approximately 25% and a fruit juice or fruit syrup concentrate of at least approximately 40% soluble solids and approximately 0% insoluble solids thereby forming a liquor having a dry weight composition of approximately 40 to 65% complex carbohydrates, approximately 35 to 55% simple sugars from the fruit juice or fruit syrup concentrate and approximately 0 to 5% nutritional components occurring naturally in the fruit juice or fruit syrup concentrate.

The sweetener composition is preferably dried to approximately 78 to 80% soluble solids to make the sweetener composition suitable for replacing corn syrup and the like or to dry the product to approximately 96 to 99% soluble solids to make the sweetener composition suitable for replacing powdered or granulated sucrose and like sweeteners. The amount of low D.E. maltodextrin is preferably between approximately 1-10% by weight, the D.E. thereof being preferably less than 5. The aforementioned fruit concentrate sweetener provides the following benefits to the FACs: a).superior humectant, b) reduced water activity factor, c) equivalent fruit servings in recommended daily allowance, d) flavor potentiator, f) an incremental level of fat mimetic equivalence, g) natural sweetener, and h) enhanced water and oil microemulsions. The fruit concentrate sweetener composition further solubilizes the glycerides selected from at least one of enzymatically modified oils, fats, and fatty acids of mono-, di-, and tri-glycerides, and lipolyzed modified oils, fats, and fatty acids of mono-, di-, and tri-glycerides. The fruit concentrate sweetener achieves superior dispersion of fats yielding an improvement in richness.

The combination of the fruit concentrate sweetener and soluble fiber, such as a dietary soluble Fibersol™ from Matsutani Chemical Industry Co. Ltd., of Itami City, Hyogo, Japan, further reduces the moisture transport from high moisture ingredients (e.g., caramels, sauces, etc.) to low moisture ingredients such as proteins in high protein bars, breads, pie crusts, and nuts. The synergistic role of the fruit concentrate sweetener and soluble fiber uniquely achieves enhancement of shelf life, maintained moisture for product freshness with good moisture release properties during consumption, and the health benefits of fiber as a probiotic. The excellent humectant properties of the fruit concentrate sweetener combined with the excellent water binding properties of soluble fiber minimize the moisture migration from high to low moisture content ingredients. A superior dispersion of fats, further reduction in water activity, functional benefits of fiber and fruit, and enhanced shelf-life is also the result of implementing the inventive combination of ingredients.

Cross-functional Flavor Potentiators and Oral Care Actives

Polyphenols play a multipurpose role in the inventive FACs. The principal incorporation of polyphenols in FACs is the flavor contribution. Polyphenols such as cocoa polyphenols (derived from cacoa (cocoa) beans, cacoa (cocoa) bean husks, and tea polyphenols are essentially flavor amplifiers. Polyphenol extracts of many other food products include grape, apple, and green tea. More recently, certain polyphenols including polyphenols present in red wine and green tea have become known to inhibit several enzymes including amylase, lipases, pectin esterases, cellulases, and beta-galactosidase. The most synergistic polyphenol and thus the preferred polyphenol is cocoa polyphenol for use in chocolate flavored FACs. Another alternative is a blend of tea polyphenols and cocoa polyphenols, whereby the tea polyphenols are a less expensive yet very effective complement to cocoa polyphenols in their ability to achieve the oral care functional benefits.

Polyphenols have another advantage in the inventive FACs, which includes the ability of polyphenols to bind themselves to mouth bacteria prior to the detrimental ability of the bacteria to form plaque. However the majority of polyphenols are not readily soluble in aqueous solvents as the dominant force is hydrophobic. Thus, specially prepared cocoa polyphenols that are water-soluble provide both cocoa flavor amplification and rapid distribution of polyphenols throughout the oral cavity. The rapid distribution of water-soluble polyphenols provide the initial bacteria destroying capabilities and flavor burst, while the oil soluble polyphenols serve as a delayed time-release reinforcement against the remaining bacteria. The net result is that the combination of oil and water phased emulsions with their respective solubilized polyphenols enable the reduction of both hydrophilic and hydrophobic bacteria, thus reducing the size and accumulation of bacterial colonies in the oral cavity. Bacteria in the mouth, specifically present on the tongue, is a primary source of protein denaturing that causes halitosis. The more preferred use of a combination of water and oil soluble polyphenols is at a combined usage level of between approximately 0.05% to approximately 2.0%, more specifically preferred from 0.1% to 0.25%.

Oral care actives utilized in FACs achieve longer-term results when combined with bioadhesives that are mucin binding. Mucins, which are glycoproteins that play a critical role in the formation of a lubricating biofilm, are capable of protecting the underlying mucosa and dental structures from both chemical and physical harm. There are two major salivary mucin glycoproteins designated MG1 and MG2. MG1 has been shown to form complexes with a range of proteins including the group of amylase, proline-rich proteins, statherin, and histatins. MG1 is most likely aimed at protecting the teeth by helping to form a low energy surface. MG2 most likely prevents colonization of microorganisms by presenting identical surface carbohydrates (as expressed in the pellicle and mucosal proteins) in the fluid phase. Histatins are considered the most important antifungal factors (Lal., et al., 1992 Oral Microbio. Immunol. 7:44-50 as cited in Dr. Ken Miyasaki's course book Chapter 12 on “Mucosal Immunity” of UCLA Dentistry (currently available on line at http://www.dent.ucla.edu/sod/courses/OB472b/Ch12.pdf)). Proline-rich proteins maintain saliva in a supersaturated state with respect to calcium phosphate. The above paragraphs are cited from Dr. Ken Miyasaki's course book Chapter 12 on “Nucosal Immunity” of UCLA Dentistry.

Lecithin, another source of proline-rich proteins, is a common emulsifier in chewing gum, confectionery, and beverage products. Unfortunately, high levels of polyphenols and lecithin (such as used in liposomes additionally) precipitate out the polyphenols thus making the polyphenols less capable of forming the protective biofilm layer. This is important for multiple reasons as the proline-rich proteins maintain calcium and phosphate in a supersaturated solution. The high concentration of calcium and phosphate ions promotes remineralization of tooth enamel. Other components present in chewing gum, confectionery, and beverage products include acids (especially in the popular sour candies). Higher pHs are typically not advantageous in promoting oral health. However, the specific use of citric acid (lemon juice contains citric acid) largely prevents the ionization of the hydroxy groups of polyphenols. Virtually un-ionized hydroxy groups restrict the interaction between the polyphenols and proteins in the mouth having both the reduction in protein-polyphenol complexation, and the reduction of the astringent taste normally associated with polyphenols.

Other means of stabilizing polyphenols from agglomeration include microencapsulation, even when the microencapsulation is not technically a continuous shell. Sugar esters through a homogenous microemulsion with a “high water solubilization fraction . . . makes it possible to immobilize water soluble materials and preserve activity or stability of active matters such as enzymes, aroma molecules, and volatile oxidation-sensitive compounds” as noted in Sugar—Ester Nonionic Microemulsion: Structural Characterization by Otto Glatter, et.al published by _____. Furthermore, increasing salt concentrations in the aqueous medium yields higher levels of polyphenols in the lipid bilayers of liposomes. This realizes another synergistic effect of flavor amplifiers such as salt that are often present with protein hydrolysates. Both the salt and protein hydrolysates potentiate a wide range of flavors, including chocolate, peanut butter, and vanilla. The use of casein and calcium phosphate that form sub-micelles sequestering the polyphenols within the casein sub-micelles, thus reducing the astringent taste, also reduce the astringent flavor of cocoa powder. The sequestering of polyphenols provides a secondary benefit of providing time delay release of the oral care polyphenol actives for enhanced long term performance and long lasting flavor infusion. A matrix of starch hydrolysate acid esters encapsulate the water insoluble polyphenols encapsulated therein, again promoting the long-term time delay effects of the polyphenols. Additionally a matrix of hydrogenated starch hydrolysate and maltodextrin serves the same purpose of having the water insoluble polyphenols encapsulated therein.

The reduction of polyphenol agglomeration is also achieved by the use of colloidal stabilizers. The preferred colloidal stabilizers include polysaccharides, and the more preferred polysaccharides include gum arabic, and pectins. Citric acid, as noted above, prevents the ionization of hydroxy groups of polyphenols, thus restricting the complexation of polyphenols and proteins. Pre-complexation reduces the subsequent complexation between polyphenols and mucousal glycoproteins in the oral cavity thus limiting the characteristic astringent taste of polyphenols, again providing a significant benefit in “flavor masking” . Complexation of polyphenols with enzymes (active proteins) has the synergistic effect of reducing the strong astringent flavor contribution of polyphenols that are present in the disclosed invention as applied to oral care even though the flavor contribution is not always desired.

Oral care products may have both breath freshness and anti-plaque requirements. The selection of specific microencapsulants that break down in the gastrointestinal tract and not in the oral cavity to encapsulate actives limit the “returned odor”, an odor that returns to the oral cavity from the gastrointestinal tract after digesting food, effectively addressing the second of the main sources of bad breath (also referred to as “halitosis”). The treatment of halitosis is often through the use of parsley oil. Polyphenols extracted from apples uniquely prevent the attachment of S. sobrinus to tooth surfaces and result in a higher level of effectiveness than parsley seed oil in controlling halitosis.

FACs incorporate at least one oral care active that realizes the benefits of dental plaque inhibiting, halitosis inhibiting, and digestive aids; natural antioxidants, said antioxidants that specifically limit formation of free radicals in the oral cavity; and metal chelators and bicarbonate. The effectiveness of polyphenols and enzymes are maximized by a wide range of means including, though not limited to encapsulation, colloidal stabilization, or pre-complexing with polyphenols and protecting enzymes against both a reduction in activity and protein denaturing.

Pre-complexing of polyphenols is preferably done with the use of proteins as detailed above in the flavor potentiator section of this application. More preferable are proteins selected from dairy proteins, and more particularly those selected from partially denatured proteins. Partially denatured proteins have an effectively “unfolded” protein structure that leads to two important benefits for FACs. The first benefit is that an unfolded protein has fat mimetic properties in addition to the flavor profile associated with the protein itself One such example is the use of a partially denatured whey protein that provides a dairy note (such as milk chocolate when in combination with cocoa, cocoa butter and its derivatives). The second benefit is the opening up of more polyphenol binding sites. Partially denatured protein enables complexation with polyphenols in order to reduce the astringent taste. Monoammonium glycyrrhizinate, a natural licorice derivative, is also another means of reducing the astringent taste of many polyphenols, especially the polyphenols present in (or extracted from) cocoa powder. This is especially important, as cocoa polyphenols turn off an enzyme responsible for converting sugar into polysaccharoses in the oral cavity. Polysaccharose is a binding agent that causes dental plaque to attach to the teeth.

Flavor potentiators as mentioned previously include enzymes, some of which are important in oral health. Enzymatic activity levels must be critically maintained (i.e., not inhibited) in order for the enzymes to remain an effective active against tooth decay and plaque buildup. The relatively high temperature conditions involved in preparing many chewing gum, confectionery, oral care and beverage products adversely affect the enzyme activity levels. Other factors that affect the enzyme activity levels include the selection of aforementioned protein hydrolysates, and polyphenols.

One such matter of affecting enzyme activity levels includes enzyme polyphenol interactions. The mechanisms involved in forming inhibited vs. stabilized humic-enzyme complexes are speculative. It is speculated that the determining factor is associated with the relationship between the enzyme and humic binding site relative to the location of the enzyme active site. Binding mechanisms that block the availability of the active site for substrate hydrolysis likely result in the complete inhibition of the enzyme. However, if the enzyme is complexed with the humic molecule such that the active site remains exposed to potential interactions with substrates, then the enzyme is stabilized, will not denature or degrade, and will remain hydrolytically active. The enzymes are preferably selected from the group of pure enzymes or an ersatz mixture of lactoperoxidase, lysozyme, salivary peroxidase, lactoferrin, proteases, lipases and/or glycohydrolases in various ratios. The extraction of enzymes by low temperature means is always preferred over drying in order to maintain high activity levels. Many enzymes are capable of grain alcohol extraction to maintain high activity levels.

Lysozyme cleaves β-1,4 linkage between N-acetylglucosamine and N-acetylmuramicacid in the peptidoglycan layer of the bacterial cell wall. Salivary peroxidase enhances the antimicrobial effects of lysozyme and lactoferrin. Lactoferrin is an iron-binding glycoprotein. Multiple mechanisms of antimicrobial activity are cited that include: a) binding of iron that is needed by all microorganisms to grow, b) prevention of electron donation by Fe(2+) to environmental hydrogen peroxide and subsequent reduction into hydroxy free radical.

Colloidal stabilization of polyphenols as a means to reduce polyphenol complexation and precipitation further reduces the agglomeration and aggregation of polyphenols into a non-dispersible precipitate. Polysaccharides are one such group of stabilizers against polyphenol precipitation. Gum arabic, and pectin are more preferred to protect polyphenols and enzymes from losses in activity levels due to precipitation. Citric acid, more preferred from lemon juice, also reduces precipitation by reducing the ionization of the hydroxy groups of the polyphenols.

The methods of encapsulating polyphenols as detailed above are also effective methods of protecting enzymes from denaturing and deactivation. Contacting the protein or enzyme with a block-wise enzymatically de-esterified pectin is also effective. Other proteins not specifically categorized as enzymes have oral health benefits. These include, though are not limited to, glycomacropeptide and cheese proteins as they both impede demineralization of tooth enamel and aid in remineralization.

Encapsulation is powerful in enhancing the performance of the inventive amplified flavors, flavor potentiators, and oral care actives. The encapsulant shell material is selected from raw materials that demonstrate at least one of the following physical characteristics that include film forming, charge polarity, hydrophobic or hydrophilic tail, microemulsions, or controlled dissolution. The method for controlled dissolution is dependent on the specific functionality of the encapsulated contents. Functional benefits for the oral cavity would preferably use an encapsulated shell that is water soluble when rapid dispersion is required. Functional benefits for the oral cavity with the objective of time released dispersion would preferably contain materials that act as bioadhesives (to bind to mucin or mucosa) and/or utilize encapsulant shell materials that are subject to enzymatic breakdown within the oral cavity. Functional benefits for the gastrointestinal tract would preferably use an encapsulated shell that is pH sensitive, whereby the specific target area is accompanied by a breakdown in a specific pH range. The additional use of bioadhesives promotes the effective lifetime of the active within the desired target area.

As a result of the varying desired functionality, release characteristics and, target areas, a wide range of acceptable encapsulant shell materials exist. The encapsulant shell material includes, though is not limited to, at least one from the group of gum arabic, fat mimetics, liposomes, liposomes in sol-gels, shellac, fats, hydrolyzed fats, ethyl cellulose, hydroxy propyl methylcellulose, starches and modified starches, polymers, waxes, alginate & alginic acid (e.g., sodium alginate), calcium caseinate, calcium polypectate, carboxyl cellulose, carrageenan, cellulose acetate phthalate, cellulose acetate trimellitate, chitosan, corn syrup solids, dextrins, fatty acids, fatty alcohols, gelatin, gellan gums, hydroxy cellulose, hydroxy ethyl cellulose, hydroxy methyl cellulose, hydroxy propyl cellulose, hydroxy propyl ethyl cellulose, hydroxy propyl methyl cellulose, hydroxy propyl methyl cellulose phthalate, lipids, liposomes, low density polyethylene, mono-, di- and tri-glycerides, pectins, phospholipids, polyethylene glycol, polylactic polymers, polylactic co-glycolic polymers, polyvinyl pyrolindone, stearic acid and derivatives, gums (e.g., xanthum) and proteins (e.g., zein, gluten).

The encapsulant shell material selection process is further based on the preferred release mechanism and the “carrier” of the dry powder. The encapsulant shell can be made water or oil soluble. The release mechanism of the encapsulant is selected from the group of mechanical rupture of the capsule shell, dissolution of the shell, melting of the shell, and diffusion of the shell. The encapsulating agents utilized to make the encapsulant shell are selected from the group of fats, hydrolyzed fats, ethyl cellulose, hydroxy propyl methylcellulose, starches and modified starches, polymers, waxes, alginate & alginic acid (e.g., sodium alginate), calcium caseinate, calcium polypectate, carboxyl cellulose, carrageenan, cellulose acetate phthalate, cellulose acetate trimellitate, chitosan, corn syrup solids, dextrins, fatty acids, fatty alcohols, gelatin, gellan gums, gum arabic, hydroxy cellulose, hydroxy ethyl cellulose, hydroxy methyl cellulose, hydroxy propyl cellulose, hydroxy propyl ethyl cellulose, hydroxy propyl methyl cellulose, hydroxy propyl methyl cellulose phthalate, lipids, liposomes, low density polyethylene, mono-, di- and tri-glycerides, pectins, phospholipids, polyethylene glycol, polylactic polymers, polylactic co-glycolic polymers, polyvinyl pyrolindone, shellac, stearic acid and derivatives, gums (e.g., xanthum) and proteins (e.g., zein, gluten).

More specifically preferred encapsulant shell materials for the encapsulation of polyphenols and enzymes are selected from the group of sugar esters, liposomes, casein and calcium phosphate, matrix of starch hydrolysate acid ester, and matrix of hydrogenated starch hydrolysate and maltodextrin.

Numerous methods of creating encapsulations are known in the industry, whereby the flavor additives, flavor potentiators, oral care actives, and amplified flavor additives are encapsulated, and incorporated in the present invention wherein the ingredients are encapsulated by a method selected from the group of dropping method filler, spray dried, fluid-bed coated, coacervation, molten encapsulating with spray chilling, and mixing of flavor enhancer with a polymer as the encapsulating agent with the resulting mixture being extruded into fine fibers, mixed with an absorbent, and included in a powder material.

The numerous benefits realized by encapsulation, specifically the time-release characteristic, enables the controlled release of the inventive flavor amplifiers and potentiators. Encapsulating ingredients from the group of glycerides selected from enzymatically modified oils, fats, and fatty acids of mono-, di-, and tri-glycerides; glycerides selected from lipolyzed modified oils, fats, and fatty acids of mono-, di-, and tri-glycerides; pervaporated flavors; high intensity sweeteners; ingredients selected from the group of colorants, salt or salt substitutes; prior encapsulated actives; and standard flavors provide long lasting flavors, staged flavor release (e.g., chocolate followed by mint flavor), and minimization of flavor volatiles during manufacturing and storage of FACs. The utilization of multiply encapsulated nutraceutical actives, pharmaceutical actives and antioxidants, flavor additives, flavor potentiators, oral care actives, and amplified flavor additives is anticipated wherein the multiply encapsulated shells are further comprised of alternating shells made from water or oil soluble ingredients that further encapsulate at a minimum the prior encapsulated actives.

The performance of enzymes, a special class of proteins, is dependent on maintaining conditions at all times from raw materials procurement to manufacturing, and from consumption to transport of enzymes to target area. The active enzymes must be stabilized in order to reduce the loss of activity and protein denaturing by further inclusion of at least one stabilizer selected from the group of lactoperoxidase, pure grain alcohol, polysaccharides, and block-wise enzymatically de-esterified pectin, sugar esters, liposomes, starch hydrolysate, and protein hydrolysate. The inclusion of said stabilizers, whether within the encapsulant shell or not encapsulated, obtains superior enzymatic activity levels.

The more specifically preferred encapsulation is further comprised of active release agents to achieve controlled release of flavor, pharmaceutical, or nutraceutical actives; bioadhesive agents to bind encapsulant shell and encapsulated actives to mucin and mucosa; and time release agents to regulate the dissolution, melting, or diffusion of the shell. The encapsulated shell is designed to achieve release of the encapsulated flavor ingredients by at least one mechanism selected from the group of mechanical rupture of the capsule shell, dissolution of the shell, melting of the shell, and diffusion of the shell.

Lastly, optimizing the performance of all flavor and oral care actives is achieved by specific manufacturing methods for protecting the actives. Specific methods of protecting the oral care actives (e.g., polyphenols and enzymes) are preferably selected from blending said actives into a composition comprised of at least powdered gum base; maintaining process temperatures below enzyme deactivation or protein denaturing temperatures; applying active enzymes as coating after any high temperature processing, such high temperature processing being above enzyme deactivation or protein denaturing temperatures; applying active enzymes as blended ingredient after any high temperature processing, such high temperature processing being above enzyme deactivation or protein denaturing temperatures; applying active enzymes as liquid center of confectionery to minimize exposure to high temperature processing, such high temperature processing being above enzyme deactivation or protein denaturing temperatures; utilizing encapsulation of active enzymes able to withstand high pressures of stamping or tabletting; utilizing multilayer encapsulation of active enzymes; utilizing enzymes in a liquid form to achieve maximum activity levels; and minimizing encapsulant shell diameter size to less than approximately 2 millimeters of encapsulated active enzymes.

Incorporation of LEMT Flavorant into Encapsulated Examples

LEMT encapsulated in a capsule shell that will rupture from mechanical pressure is incorporated into the unfinished FAC in an amount of approximately 100% of the desired flavor associated with the LEMT.

LEMT encapsulated in a capsule shell that will rupture from mechanical pressure is incorporated into the unfinished FAC in an amount of approximately 80% of the desired flavor associated with the LEMT. LEMT encapsulated in a capsule shell that will diffuse through the shell when chewed is incorporated into the unfinished FAC in an amount of approximately 20% of the desired flavor associated with the LEMT.

LEMT encapsulated in a capsule shell that will rupture from mechanical pressure is incorporated into the unfinished FAC in an amount of approximately 60% of the desired flavor associated with the LEMT. LEMT encapsulated in a capsule shell that will diffuse through the shell when chewed is incorporated into the unfinished FAC in an amount of approximately 20% of the desired flavor associated with the LEMR. LEMT not encapsulated is incorporated directly into the unfinished FAC in an amount of approximately 20% of the desired flavor associated with the LEMT for an immediate flavor release.

LEMT encapsulated in a capsule shell that will rupture from mechanical pressure is incorporated into the unfinished FAC in an amount of approximately 60% of the desired flavor associated with the LEMT. LETff encapsulated in a capsule shell that will dissolute the shell when chewed is incorporated into the unfinished FAC in an amount of approximately 20% of the desired flavor associated with the LEMT. LEMT not encapsulated is incorporated directly into the unfinished FAC in an amount of approximately 20% of the desired flavor associated with the LEMT for an immediate flavor release.

In an embodiment, the FAC flavors in a wide range of flavors are now possible. The flavor range includes individual flavors and combinations of flavors.

In an embodiment, the FAC flavor is chocolate. Such flavor is comprised of LEMT cocoa butter and chocolate solids.

In an embodiment, the FAC flavor is milk chocolate. Such flavor is comprised of LEMT cocoa butter, chocolate solids, and dry milk powder.

In an embodiment, the FAC flavor is chocolate mint. Such flavor is comprised of LEMT cocoa butter, chocolate solids, and mint.

In an embodiment, the FAC flavor is beer. Such flavor is comprised of LEMT cocoa butter, and dry beer solids.

In an embodiment, the FAC flavor is nacho. Such flavor is comprised of LEMT cheese, dry solid spices (e.g., jalapeno pepper, yeast, etc.) and dry beer solids.

In an embodiment, the FAC flavor is butter popcorn. Such flavor is comprised of LEMT butter, LEMT corn oil, dry solid spices and dry beer solids.

In an embodiment, the FAC flavor is peanut butter and jelly. Such flavor is comprised of LEMT peanut, dry solid spices, and dry solid fruit concentrates.

In an embodiment, the FAC flavor is complimented with other flavors. Such flavors are comprised of coffee and vanilla.

If desired, the FAC can also include cellulose fiber in an amount of up to approximately 3% of the water content, the cellulose fibers having a particle size preferably in the range of not greater than approximately 400 microns, and more preferably in the range of approximately 50-200 microns. A particle size of more than approximately 400 microns gives the gum a texture that is too gritty or stringy. The amount of cellulose fiber depends to a large extent on the amount of water in the gum. Gums having approximately 15% of water require no cellulose fibers or at most a very small amount, while gums having a high water content of up to approximately 60% require higher cellulose fiber content in a range of approximately 0.05-3% of the water content. If less than approximately 0.05% cellulose fiber is used in such gums, the amount of fiber is not sufficient to provide the desired texture for good chewability. On the other hand, an amount of more than approximately 3% makes the product too stiff and therefore loses chewability. The higher percentage is used for higher water contents. When used at the preferred levels, the cellulose fibers serve as non-caloric moisture binders and improve the texture and mouthfeel of the gums. The cellulose fibers also improve the flavor impact in spreads having high water content. In accordance with a preferred embodiment of the present invention, the water-soluble starter distillate and oil soluble lipolyzed oils are both spray dried and in this dry form mixed with the aqueous phase from which the water-in-oil emulsion of the FAC is made.

The flavor must be homogeneously distributed within the emulsion and yet must be available to the taste buds during consumption. Still further, the flavor must also be stable upon storage of FAC for periods of at least six months and must be stable upon heating and during processing. The resulting flavor base becomes shelf stable and does not change upon storage and still further the flavor base can be more easily incorporated into the FAC since it can be mixed into the water phase and thoroughly emulsified therewith before being added to the fat phase.

The water phase can also be distributed through the oil phase by emulsification with any of the common commercial emulsifying agents such as lecithin, to form a paste, which paste can also be used for the purposes of the present invention.

Typical FAC ingredients include: gum bases (including elastomers; elastomer plasticizers; fillers; and softeners; waxes); antioxidants; colorants; flavors; and bulk sweeteners; high intensity sweeteners; flavoring agents; softeners; emulsifiers; colors; acidulants; fillers; and other components that provide desired attributes. FACs generally consists of a water insoluble portion, a water-soluble portion, and flavors. Traditionally, the water-soluble portion dissipates with a portion of the flavor over a period of time during chewing. An alternative method is to encapsulate a portion of the flavor with an encapsulant for timed release of the flavor.

The resultant FAC of the present invention will deliver more intense flavors, new flavorants derived from natural ingredients without severely affecting the texture as compared to typical non-FAC foods.

Gum Base

Standard chewing gum additives are selected from the group of group elastomers; elastomer plasticizers; fillers; softeners; waxes; antioxidants; colorants; emulsifiers; colors; acidulants; texturing agents; and other components that provide desired attributes and adding time release components selected from the group of flavors; flavoring agents; colors; acidulants; minerals, vitamins and bioactive agents.

The present invention provides copolymers with improved elastomeric behavior compounded with components selected from the group of enzymatically modified fats, oils and triglycerides, an encapsulating agent, and naturally flavored with dairy fats and creams, vegetable oils, nut oils and other triglycerides, methods for making same, and methods for making such copolymer into a chewing gum base. The additional method of use as a component in a chewing gum base and its preferred embodiments of the invention will now be set forth.

The present invention does not yet further preclude a chewing gum base composition that incorporates encapsulates of flavor and colorant agents as a means to enhance the consumer appeal due to unique flavor and colorant release characteristics as compared to typical compositions derived. Typical gum flavors and coloring are quickly dissipated during the process of chewing. It is therefore an object of the invention for a chewing gum base to release flavors and/or colorant agents over the duration of the chewing experience. The process includes the controlled flavor and active release over an extended duration for either longer lasting flavors and actives or the lower level of expensive flavors and actives.

The gum base is then further compounded with ingredients selected from the group of fats, oils, flavorants, pharmaceutical actives, nutraceutical actives, and nutrients; preferred fats, oils, flavorants, pharmaceutical actives, nutraceutical actives, and nutrients which are further encapsulated to achieve the purpose selected from the group of time release, mask off-flavors, and protection from premature breakdown in mouth, digestive tract, and stomach;

Chewing Gum

The principle of manufacturing chewing gum, wherein chewing gum as used in the context of the present invention also includes bubble gum, consists of the manufacture of its two major components: 1) the chewing gum base and 2) a non-masticatory part. The non-masticatory part consists mainly of sweeteners, softeners and flavor ingredients. The inventive chewing gum is comprised of chewing gum base, a non-masticatory part and components selected from the group of enzymatically modified fats, oils and triglycerides, an encapsulating agent, and naturally flavored with dairy fats and creams, vegetable oils, nut oils and other fatty acids of mono-, di-, and tri-glycerides and other additional gum components known in the art. The major gum components known in the art include the following ingredients: sweeteners, high intensity sweeteners, flavorants and colorants, actives, encapsulated actives, and acidulants.

The sweeteners and high intensity sweeteners comprise one or more components selected from the group of flavors, bulk sweeteners, high intensity sweeteners, low caloric bulking agents and combinations thereof, preferred bulk sweeteners are selected from the group of sugar sweeteners, sugarless sweeteners, and combinations thereof, preferred high intensity sweeteners are selected from the group of artificial sweeteners, natural intense sweeteners, and peptide sweeteners; more preferred high intensity artificial sweeteners are subjected to encapsulation by such techniques selected from the group of wet granulation, wax granulation, spray drying, spray chilling, fluid bed coating, coacervation, fiber extension, micro-encapsulation, cyclodextrin “encapsulation”; wherein exemplary sugar sweeteners are saccharides (e.g., sucrose, dextrose, maltose, dextrin, dried invert sugar, fructose, levulose, galactose, corn syrup solids); wherein exemplary sugarless sweeteners are sugar alcohols (e.g., erythritol, sorbitol, mannitol, xylitol, hydrogenated starch hydrolysates, maltitol), wherein exemplary high intensity artificial sweeteners are sucralose, aspartame, salts of acesulfame, alitame, thaumatin, saccharin and its salts, cyclamic acid and its salts, glycyrrhizin, dihydrochalcones, thaumatin, monellin, and where as exemplary low caloric bulking agents are: polydextrose; oligofructose; fructooligosaccharide; palatinose oligosaccharide; natural carbohydrate gum hydrolysate; or indigestible dextrins.

The flavorants and colorants in gum, for exemplary purposes that impart specific flavor profiles, and removing or masking undesired characteristics are cocoa powder; heat-modified amino acids; partially defatted proteins, vegetable extracts, natural extracts (e.g., citrus oils, fruit essences, peppermint oil, spearmint oil, clove oil, oil of wintergreen, anise), artificial flavorants and combinations thereof; exemplary colorants are FD&C type lakes, plant extracts, fruit and vegetable extracts and titanium dioxide and combinations thereof; preferred flavorants and colorants levels are present from approximately 0% to approximately 15% by weight, and are more fully and evenly released by the gum base of the present invention.

The gum mass also comprises one or more components selected from the group of pharmaceutical actives, nutraceutical actives, minerals, and vitamins. The actives are comprised of one or more components selected from the group of encapsulated pharmaceutical actives, nutraceutical actives, minerals, and vitamins to make an actives release chewing gum, wherein term “active release” refers to a gum that provides a controlled release of flavor, pharmaceutical, or nutraceutical actives.

The gum mass also includes acidulants as flavors and flavor enhancers selected from the group of edible acids; the preferred acids including acetic, citric, lactic, and ascorbic acid.

### EXAMPLES

### Example 1.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cocoa butter, approximately 0.5 gram of 99% glycerol and approximately 0.6 gram of mint flavor are subsequently added and mixed for approximately 5 additional minutes.

### Example 2.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cocoa butter that is encapsulated in a capsule shell and will rupture from mechanical pressure, approximately 0.5 gram of 99% glycerol and approximately 0.6 gram of mint flavor are subsequently added and mixed for approximately 5 additional minutes.

### Example 3.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 1.5 grams of LEMT cocoa butter that is encapsulated in a capsule shell that will rupture from mechanical pressure, approximately 0.5 gram of LEMT cocoa butter that is encapsulated in a capsule shell that will diffuse through the shell when chewed, approximately 0.5 gram of 99% glycerol and approximately 0.6 gram of mint flavor are subsequently added and mixed for approximately 5 additional minutes.

### Example 4.

Approximately nineteen (19) grams of gum base were added to a small gum mixer set at approximately 50° C. for a. duration of approximately 20 minutes in addition to approximately 60 grams of 6× sugar and approximately 20 grams of 45Be corn syrup. Approximately 1.5 grams of LEMT cocoa butter that is encapsulated in a capsule shell that will rupture from mechanical pressure, approximately 0.5 gram of LEMT cocoa butter that is encapsulated in a capsule shell that will diffuse through the shell when chewed, approximately 1.5 grams of LEMT cocoa butter that is not encapsulated, approximately 0.5 gram of 99% glycerol and approximately 0.6 gram of mint flavor are subsequently added and mixed for approximately 5 additional minutes.

### Example 5.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cocoa butter, approximately 2 grams of chocolate solids, approximately 0.5 gram of 99% glycerol and approximately 0.2 gram of mint flavor are subsequently added and mixed for approximately 5 additional minutes.

### Example 6.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cocoa butter, approximately 2 grams of chocolate solids, approximately 2 grams of dry milk powder, approximately 0.5 g of 99% glycerol and approximately 0.2 grams of mint flavor are subsequently added and mixed for approximately 5 additional minutes.

### Example 7.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cocoa butter, approximately 2 grams of dry beer solids, and approximately 0.5 gram of 99% glycerol are subsequently added and mixed for approximately 5 additional minutes.

### Example 8.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT cheese, approximately 2 grams of dry beer solids, and approximately 0.5 g of 99% glycerol are subsequently added and mixed for approximately 5 additional minutes.

### Example 9.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT butter, approximately 2 grams of LEMT corn oil, and approximately 0.5 gram of 99% glycerol are subsequently added and mixed for approximately 5 additional minutes.

### Example 10.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 2 grams of LEMT peanut oil, approximately 2 grams of LEMT cocoa butter, approximately 2 grams of dry grape solids and approximately 0.5 gram of 99% glycerol are subsequently added and mixed for approximately 5 additional minutes.

### Example 11.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 1.5 grams of LEMT cocoa butter that is encapsulated in a capsule shell that will rupture from mechanical pressure, approximately 0.5 gram of LEMT cocoa butter that is encapsulated in a capsule shell that will diffuse through the shell when chewed, approximately 0.5 gram of 99% glycerol and approximately 0.6 gram of vanilla flavor are subsequently added and mixed for approximately 5 additional minutes.

### Example 12.

Approximately nineteen (19) grams of gum base were added to a small gum mixer set at approximately 50° C. for a duration of approximately 20 minutes in addition to approximately 60 grams of 6× sugar and approximately 20 grams of 45Be corn syrup. Approximately 1.5 grams of LEMT cocoa butter that is encapsulated in a capsule shell that will rupture from mechanical pressure, approximately 0.5 gram of LEMT cocoa butter that is encapsulated in a capsule shell that will diffuse through the shell when chewed, approximately 0.5 gram of 99% glycerol and approximately 0.6 gram of coffee flavor are subsequently added and mixed for approximately 5 additional minutes.

### Example 13.

Approximately nineteen (19) grams of gum base were added to a small gum mixer set at approximately 50° C. for a duration of approximately 20 minutes in addition to approximately 60 grams of 6× sugar and approximately 20 grams of fruit sweetener composition. Approximately 2 grams of LEMT cocoa butter that is encapsulated in a capsule shell that will rupture from mechanical pressure, approximately 0.5 gram of 99% glycerol and approximately 0.6 gram of mint flavor are subsequently added and mixed for approximately 5 additional minutes. The fruit concentrate sweetener composition comprises a blend of a hydrolyzed starch having a dextrose equivalent (D.E.) of approximately 25 and a fruit juice or fruit syrup concentrate of approximately 40% soluble solids and approximately 2% insoluble solids thereby having a dry weight composition of approximately 50 complex carbohydrates, approximately 40% simple sugars from the fruit juice or fruit syrup concentrate and approximately 3% nutritional components occurring naturally in the fruit juice or fruit syrup concentrate. The sweetener composition was dried to approximately 80% soluble solids to make the sweetener composition suitable for replacing corn syrup.

What is claimed is:

1) A chewing gum or oral care composition having flavor profiles of increased intensity, said flavor profiles being comprised of at least one flavor ingredient selected from the group of:
enzymatically modified oils, fats, and fatty acids of mono-, di-, and tri-glycerides;
lipolyzed modified oils, fats, and fatty acids of mono-, di-, and tri-glycerides;
protein hydrolysate and its derivatives;
enzymatically modified protein powder and hydrolysate products;
fermented protein products; and
pervaporated flavors.

2) A chewing gum or oral care composition is comprised of at least one oral care active selected from the group of:
encapsulated, colloidal stabilized, or complexed polyphenols;
enzymes stabilized against reduction in activity and protein denaturing, said enzymes selected from the group of dental plaque inhibiting, halitosis inhibiting, and digestive aid enzymes; and
natural antioxidants, said antioxidants specifically limiting formation of free radicals in the oral cavity.

3) A confectionery, beverage, or oral care composition having flavor profiles of increased intensity, said flavor profiles being comprised of at least one flavor ingredient from the group of pervaporated flavors; enzymatically modified oils, fats, and fatty acids of mono-, di-, and tri-glycerides; and lipolyzed modified oils, fats, and fatty acids of mono-, di-, and tri-glycerides; combined with at least one flavor ingredient from the group of protein hydrolysate and its derivatives, enzymatically modified protein powder, hydrolysate products, and fermented protein products.

4) The chewing gum or oral care composition of claim 1 having flavor profiles of increased intensity, said flavor profiles further being comprised of at least one flavor ingredient selected from the group of: fat mimetics to modify the release of flavors and flavor enhancers in the mouth and to emulate fat mouthfeel.

5) The chewing gum or oral care composition of claim 2 having flavor profiles of increased intensity, said flavor profiles further being comprised of at least one flavor ingredient selected from the group of: fat mimetics to modify the release of flavors and flavor enhancers in the mouth and to emulate fat mouthfeel.

6) The confectionery, beverage, or oral care composition of claim 3 having flavor profiles of increased intensity, said flavor profiles further being comprised of at least one flavor ingredient selected from the group of: fat mimetics for modifying the release of flavors and flavor enhancers in the mouth and for emulating fat mouthfeel.

7) The chewing gum or oral care composition of claim 1 being further comprised of at least one flavor ingredient selected from the group of:
partially denatured protein; and
monoammonium glycyrrhizinate.

8) The chewing gum or oral care composition of claim 2 being further comprised of at least one flavor ingredient selected from the group of:
partially denatured protein; and
monoammonium glycyrrhizinate.

9) The chewing gum or oral care composition of claim 1 being further comprised of a fruit concentrate sweetener as humectant that comprises a blend of:
hydrolyzed starch having a dextrose equivalent (D.E.) of up to approximately 25;
fruit juice or fruit syrup concentrate of at least approximately 40% soluble solids and approximately 0% insoluble solids thereby forming a liquor having a dry weight composition of approximately 40 to approximately 65% complex carbohydrates;
approximately 35 to approximately 55% simple sugars from the fruit juice or fruit syrup concentrate; and
approximately 0 to approximately 5% nutritional components occurring naturally in the fruit juice or fruit syrup concentrate.

10) The chewing gum or oral care composition of claim 2 being further comprised of a fruit concentrate sweetener as humectant that comprises a blend of:
hydrolyzed starch having a dextrose equivalent (D.E.) of up to approximately 25;
fruit juice or fruit syrup concentrate of at least approximately 40% soluble solids and approximately 0% insoluble solids thereby forming a liquor having a dry weight composition of approximately 40 to approximately 65% complex carbohydrates;
approximately 35 to approximately 55% simple sugars from the fruit juice or fruit syrup concentrate; and
approximately 0 to approximately 5% nutritional components occurring naturally in the fruit juice or fruit syrup concentrate.

11) The confectionery, beverage, or oral care composition of claim 3 being further comprised of a fruit concentrate sweetener as humectant that comprises a blend of:
hydrolyzed starch having a dextrose equivalent (D.E.) of up to approximately 25;
fruit juice or fruit syrup concentrate of at least approximately 40% soluble solids and approximately 0% insoluble solids thereby forming a liquor having a dry weight composition of approximately 40 to approximately 65% complex carbohydrates;
approximately 35 to approximately 55% simple sugars from the fruit juice or fruit syrup concentrate; and
approximately 0 to approximately 5% nutritional components occurring naturally in the fruit juice or fruit syrup concentrate.

12) The chewing gum or oral care composition of claim 1 having an encapsulant shell that encapsulates at least one from the group of:
glycerides selected from enzymatically modified oils, fats, and fatty acids of mono-, di-, and tri-glycerides;
glycerides selected from lipolyzed modified oils, fats, and fatty acids of mono-, di-, and tri-glycerides;
pervaporated flavors;
high intensity sweeteners;
ingredients selected from the group of colorants, nutraceutical actives, pharmaceutical actives and antioxidants;
salt or salt substitutes;
active release agents to achieve controlled release of flavor, pharmaceutical, or nutraceutical actives;
bioadhesive agents to bind the encapsulant shell and encapsulated actives to mucin and mucosa;
time release agents to regulate the dissolution, melting, or diffusion of the shell;
prior encapsulated actives; and
standard flavors.

13) The chewing gum or oral care composition of claim 2 having an encapsulant shell that encapsulates at least one from the group of:
glycerides selected from enzymatically modified oils, fats, and fatty acids of mono-, di-, and tri-glycerides;
glycerides selected from lipolyzed modified oils, fats, and fatty acids of mono-, di-, and tri-glycerides;
pervaporated flavors;
high intensity sweeteners;
ingredients selected from the group of colorants, nutraceutical actives, pharmaceutical actives and antioxidants;
salt or salt substitutes;
active release agents to achieve controlled release of flavor, pharmaceutical, or nutraceutical actives;
bioadhesive agents to bind encapsulant shell and encapsulated actives to mucin and mucosa;
time release agents to regulate the dissolution, melting, or diffusion of the shell;
prior encapsulated actives; and
standard flavors.

14) The confectionery, beverage, or oral care composition of claim 3 having an encapsulant shell that encapsulates at least one from the group of:
glycerides selected from enzymatically modified oils, fats, and fatty acids of mono-, di-, and tri-glycerides;
glycerides selected from lipolyzed modified oils, fats, and fatty acids of mono-, di-, and tri-glycerides;
pervaporated flavors;
high intensity sweeteners;
ingredients selected from the group of colorants, nutraceutical actives, pharmaceutical actives and antioxidants;
salt or salt substitutes;
active release agents to achieve controlled release of flavor, pharmaceutical, or nutraceutical actives;
bioadhesive agents to bind encapsulant shell and encapsulated actives to mucin and mucosa;
time release agents to regulate the dissolution, melting, or diffusion of the shell;
prior encapsulated actives; and
standard flavors.

15) The chewing gum or oral care composition of claim 1 being further comprised of oils and fats in the triglyceride form processed by enzymatic interesterification.

16) The confectionery, beverage, or oral care composition of claim 3 being further comprised of enzymatic interesterified oil and fats selected from at least one from the group of oils and fats in the triglyceride form with a minimum of approximately 10% on a weight basis of one or more of omega-3, and omega-9 polyunsaturated fatty acids, and eicosapentaenoic acid and docosahexaenoic acid.

17) The chewing gum or oral care composition of claim 1 being further comprised of at least one enzyme processed to protect actives from harsh manufacturing conditions as selected from:
blending said enzymes into a composition comprised of at least powdered gum base;
maintaining process temperatures below enzyme deactivation or protein denaturing temperatures;
applying active enzymes as coating after any high temperature processing, said high temperature processing being above enzyme deactivation or protein denaturing temperatures;
applying active enzymes as blended ingredient after any high temperature processing, said high temperature processing being above enzyme deactivation or protein denaturing temperatures;
applying active enzymes as liquid center to minimize exposure to high temperature processing, said high temperature processing being above enzyme deactivation or protein denaturing temperatures;
utilizing encapsulation of active enzymes means able to withstand high pressures of stamping or tabletting;
utilizing multilayer encapsulation of active enzymes;
utilizing enzymes in a liquid form to achieve maximum activity levels; and
minimizing encapsulant shell diameter size to less than approximately 2 millimeters of encapsulated active enzymes.

18) The confectionery, beverage, or oral care composition of claim 3 being further comprised of at least one enzyme processed to protect actives from harsh manufacturing conditions as selected from:
maintaining process temperatures below enzyme deactivation or protein denaturing temperatures;
applying active enzymes as coating after any high temperature processing, said high temperature processing being above enzyme deactivation or protein denaturing temperatures;
applying active enzymes as blended ingredient after any high temperature processing, said high temperature processing being above enzyme deactivation or protein denaturing temperatures;
applying active enzymes as liquid center to minimize exposure to high temperature processing, said high temperature processing being above enzyme deactivation or protein denaturing temperatures;
utilizing encapsulation of active enzymes means able to withstand high pressures of stamping or tabletting;
utilizing multilayer encapsulation of active enzymes;
utilizing enzymes in a liquid form to achieve maximum activity levels; and
minimizing encapsulant shell diameter size to less than approximately 2 millimeters of encapsulated active enzymes.

19) The enzymatically modified oils, fats, and fatty acids of mono-, di-, and tri-glycerides, and lipolyzed modified oils, fats, and fatty acids of mono-, di-, and tri-glycerides of claim 1 being selected from the group of cocoa butter, peanut oils, dairy fats and creams including butter, cheese, milk, vegetable oils, nut oils, and natural oils from spices and fruits, and other triglycerides.

20) The enzymatically modified oils, fats, and fatty acids of mono-, di-, and tri-glycerides, and lipolyzed modified oils, fats, and fatty acids of mono-, di-, and tri-glycerides of claim 3 being selected from the group of cocoa butter, peanut oils, dairy fats and creams including butter, cheese, milk, vegetable oils, nut oils, and natural oils from spices and fruits, and other triglycerides.

21) The encapsulant shell of claim 12 being comprised of alternating shells of water or oil soluble ingredients that further encapsulate at a minimum the prior encapsulated actives.

22) The encapsulant shell of claim 12 being comprised of shell ingredients to achieve release of the encapsulated flavor ingredients by at least one mechanism selected from the group of mechanical rupture of the capsule shell, dissolution of the shell, melting of the shell, and diffusion of the shell.

23) The fruit concentrate sweetener composition of claim 9 further solubilizing the glycerides selected from at least one of enzymatically modified oils, fats, and fatty acids of mono-, di-, and tri-glycerides, and lipolyzed modified oils, fats, and fatty acids of mono-, di-, and tri-glycerides.

24) The fruit concentrate sweetener composition of claim 23 being further processed with steps selected from the group:
dried to approximately 78 to approximately 80% soluble solids to make the sweetener composition suitable for replacing corn; and
dried to approximately 96 to approximately 99% soluble solids to make the sweetener composition suitable for replacing powdered or granulated sucrose.

25) The protein hydrolysate and its derivatives, enzymatically modified protein powder, hydrolysate products, and fermented protein products of claim 1 being further selected from at least one of the group of:
soy powder and its derivatives, soy protein hydrolysate and its derivatives, enzymatically modified soy powder, hydrolysate products and fermented soy protein products;
wheat protein hydrolysate and its derivatives, enzymatically modified wheat powder and hydrolysate products and fermented wheat protein products; and
milk protein hydrolysate and its derivatives, enzymatically modified milk powder and hydrolysate products and fermented milk protein products.

26) The protein hydrolysate and its derivatives, enzymatically modified protein powder, hydrolysate products, and fermented protein products of claim 3 being further selected from at least one of the group of:
soy powder and its derivatives, soy protein hydrolysate and its derivatives, enzymatically modified soy powder, hydrolysate products and fermented soy protein products;
wheat protein hydrolysate and its derivatives, enzymatically modified wheat powder and hydrolysate products and fermented wheat protein products; and
milk protein hydrolysate and its derivatives, enzymatically modified milk powder and hydrolysate products and fermented milk protein products.

27) The fat mimetic of claim 4 being further selected from the group of cocoa, partially defatted nut flour, defatted nut flour, food starch, modified food starch, protein, liposome, chitosan, peptide, fiber, cellulose, lecithin and interesterified triglycerides.

28) The fat mimetic of claim 6 being further selected from the group of cocoa, partially defatted nut flour, defatted nut flour, food starch, modified food starch, protein, liposome, chitosan, peptide, fiber, cellulose, lecithin and interesterified triglycerides.

29) The fat mimetic of claim 4 wherein the fat mimetic powder comprises particles having an average size from approximately 100 nanometers to approximately 10,000 nanometers.

30) The fat mimetic of claim 6 wherein the fat mimetic powder comprises particles having an average size from approximately 100 nanometers to approximately 10,000 nanometers.

31) The fat mimetic of claim 4 wherein the fat mimetic powder is produced by at least one process selected from the group of sonication, hydrodynamic cavitation, and cryogenic grinding.

32) The fat mimetic of claim 6 wherein the fat mimetic powder is produced by at least one process elected from the group of sonication, hydrodynamic cavitation, and cryogenic grinding.

33) The polyphenols of claim 2 being selected from a combination of at least one water-soluble and at least one oil soluble polyphenol to reduce both hydrophilic and hydrophobic bacteria.

34) The polyphenols of claim 2 being selected from polyphenol extracts of cocoa, cocoa bean husk, apple, grape, and tea.

35) The polyphenols of claim 2 being further mixed with citric acid to prevent the ionization of the hydroxy groups of polyphenols.

36) The polyphenols of claim 2 being further mixed with bioadhesive agents to further promote the binding to mucin and mucosa.

37) The encapsulated polyphenols of claim 2 being further comprised of at least one encapsulant selected from the group of sugar esters, liposomes, casein and calcium phosphate, matrix of starch hydrolysate acid ester, and matrix of hydrogenated starch hydrolysate and maltodextrin.

38) The colloidal stabilized polyphenols of claim 2 being further comprised of at least one stabilizer selected from the group of polysaccharides, and citric acid.

39) The complexed polyphenols of claim 2 being further comprised of at least one complexing agent selected from the group of dairy proteins, and partially denatured proteins.

40) The enzymes stabilized against reduction in activity and protein denaturing of claim 2 being further comprised of at least one stabilizer selected from the group of lactoperoxidase, pure grain alcohol, polysaccharides, and block-wise enzymatically de-esterified pectin, sugar esters, liposomes, starch hydrolysate, and protein hydrolysate.

41) The enzymes of claim 18 being further comprised of at least one stabilizer selected from the group of lactoperoxidase, pure grain alcohol, polysaccharides, and block-wise enzymatically de-esterified pectin, sugar esters, liposomes, starch hydrolysate, and protein hydrolysate.

42) The polyphenols of claim 2 being further comprised of at least one stabilizer preferably selected from at least one from the group of gum arabic, and pectin.

43) The confectionery, beverage, or oral care composition of claim 3 being further comprised of a soluble fiber to minimize the moisture migration between high to low moisture content ingredients.

44) The oral care product comprised of oral care compositions of claim 1 including chewing gum, mints, toothpaste, mouthwash, breathstrips, edible films, liquid concentrate drops, and soft capsules.

45) The oral care product comprised of oral care compositions of claim 2 including chewing gum, mints, toothpaste, mouthwash, breathstrips, edible films, liquid concentrate drops, and soft capsules.

46) The confectionery, beverage, or oral care products comprised of confectionery, beverage, or oral care compositions of claim 3 including cookies, bars, fudge, caramels, taffy, mints, chocolates, butters, spreads, chewing gum, gummy products, dry powder mixes, carbonated drinks, dairy milk and yogurt drinks, non-dairy milk and yogurt drinks, chocolate drinks, coffees, and teas.
